(19) United States
(12) Patent Application Publication
(10) Pub. No.: US 2013/0072732 A1
(43) Pub. Date: Mar. 21, 2013

(54) METHOD OF SEPARATING BUTENE-2 FROM A C4 CUT CONTAINING BUTENE-2 AND BUTENE-1 BY SELECTIVE OLIGOMERIZATION OF BUTENE-1

(71) Applicant: IFP ENERGIES NOUVELLES, Rueil-Malmaison Cedex (FR)

(72) Inventors: Pierre Alain BREUIL, Lyon (FR); Helene OLIVIER-BOURBIGOU, Saint Genis-Laval (FR)

(73) Assignee: IFP ENERGIES NOUVELLES, RUEIL-MALMAISON CEDEX (FR)

(21) Appl. No.: 13/622,476

(22) Filed: Sep. 19, 2012

(30) Foreign Application Priority Data
Sep. 20, 2011 (FR) 11/02.849

(51) Int. Cl. C07C 7/177

(52) U.S. Cl. 585/251; 585/850; 585/809

(57) ABSTRACT

The present invention describes a method of separating butene-2 from a C4 cut containing butene-2 and butene-1 by selective oligomerization of butene-1 to predominantly linear oligomers. Optional additional steps make it possible to separate isobutene, 1,3-butadiene as well as traces of acetylene hydrocarbons optionally present in the C4 feedstock.

The present invention describes a method of separating butene-2 from a C4 cut containing butene-2 and butene-1 by selective oligomerization of butene-1 to predominantly linear oligomers in the presence of an iron-based catalytic composition. Optional additional steps make it possible to separate isobutene, 1,3-butadiene as well as traces of acetylenic hydrocarbons optionally present in the C4 feedstock.

C4 cuts are available in large quantities in refineries and petrochemical plants. They are produced by the standard processes such as catalytic cracking or steam cracking. C4 cuts can also be obtained from biomass after dehydration of alcohols. These C4 cuts typically contain olefinic compounds such as n-butenes (butene-1 and butenes-2), isobutene and optionally paraffins (isobutane and n-butane), dienes (such as 1,3-butadiene) as well as acetylenic impurities. Table 1 shows the typical compositions of C4 cuts originating from fluidized-bed catalytic cracking, from steam cracking and from dehydration of alcohols.

TABLE 1
Typical compositions of C4 cuts

| | Typical composition | | Composition |
|---|---|---|---|
| | (wt. %) | | (wt. %) |
| | fluidized-bed catalytic | Composition (wt. %) | Dehydration of |
| | cracking * | Steam cracking * | alcohols |
| n-Butane | 11 | 5 | — |
| Butene-1 | 13 | 28 | 20 |
| Butene-2 | 26 | 20 | 20 |
| Isobutene | 15-25 | 45-50 | 40 |
| Isobutane | 35 | 2 | — |
| Butadiene | 0.5 | 1 | — |

* after selective hydrogenation

Whatever process is used, their separation is still often problematic. Given the small difference in boiling points of these compounds (see Table 2), it is difficult to separate them by distillation, and in particular butene-1 and isobutene cannot be separated by distillation. This is why more effective and selective physical and chemical separation techniques must be implemented.

TABLE 2
Boiling point of C4 compounds:

| | Boiling point (° C.) |
|---|---|
| n-Butane | −0.4 |
| Butene-1 | −6.3 |
| Butene-2 Cis | 3.7 |
| Butene-2 Trans | 0.9 |
| Isobutene | −6.9 |
| Isobutane | −11.7 |

Industrially, separation of the C4 cut generally begins with the separation of butadiene, for example by liquid-liquid extraction with a solvent (N-methyl-2-pyrrolidone, DMF, acetone, acetonitrile etc.) and/or by selective hydrogenation making it possible to obtain a mixture of isobutenes, n-butenes and butanes. Isobutene is then separated from the other C4 compounds since it is branched and it has a different reactivity. The separation of isobutene is carried out by circuitous routes, converting it by processes such as etherification with methanol or selective oligomerization or polymerization leading respectively most typically to MTBE (methyl tert-butyl ether), to diisobutene or PIB (polyisobutene). The routes used depend on the market outlets. Pure isobutene can be regenerated from MTBE by cracking. Thus, patent EP0869107 describes a method of producing alpha-olefins (butene-1), tertiary olefin (isobutene) and/or ether from an unsaturated hydrocarbon cut.

After removal of isobutene, only the n-butenes and the butanes remain. A further separation is not generally carried out, since the alkanes do not react during the reactions for processing butenes and are therefore extracted as inert materials. In principle, separation of butene-2 (cis and trans) from butene-1 by distillation is possible but is still difficult. The butanes can be separated from the n-butenes by extractive distillation.

Other developments allow more extensive separations of the C4 cut, and in particular separation of the n-butenes. These methods often require several steps. Thus, patent EP0079679 describes a method of separating butene-1 from butene-2 in two distillation steps plus two extractive distillation steps.

All the butenes are useful raw materials in industrial synthesis.

The butene-2 contained in the C4 cuts can be used as substrate for the production of trimethylpentenes (isooctanes), used as high-octane additives for fuel, by an alkylation process with isobutane.

It can also be used in combination with ethylene for the production of propylene, implementing a cross-metathesis reaction. For example, patent U.S. Pat. No. 5,898,091 describes the combined production of propylene and ether from a C4 cut by a process implementing the following different steps: 1) hydroisomerization of the dienes, 2) etherification of isobutene to MTBE, 3) separation of the oxygen-containing compounds, 4) metathesis of butene-2 with ethylene. Patent U.S. Pat. No. 6,207,115 describes the combined production of propylene and polyisobutene by a process implementing the following steps: 1) hydroisomerization of the dienes, 2) polymerization of isobutene, 3) metathesis of butene-2 with ethylene.

The linear butenes obtained from the C4 cut can also be used for the production of predominantly branched octenes by an oligomerization process (Dimersol) or for the production of sec-butanol and methyl ethyl ketone (MEK).

Furthermore, there are processes for the oligomerization of linear alpha-olefins. Patent US2002/0177744 describes the synthesis of butene-1 dimers and other linear alpha-olefins by coupling of alpha-olefins catalyzed by iron/cobalt complexes activated with an aluminum derivative. The C8 dimers formed (octenes) have the particular feature that they have very good linearity.

The predominantly linear octenes can be used advantageously for the synthesis of aldehydes or alcohols after hydroformylation and hydrogenation. These alcohols can be used for example in the production of phthalates (plasticizers) with improved properties.

It has now been found that these iron complexes could be used for selectively converting butene-1 by an oligomerization reaction when the latter is mixed with a feedstock containing butene-2 and optionally isobutane, n-butane and/or isobutene.

The oligomerization of light olefins (C2-C4) by transition metal complexes is mainly described in the literature in the case of ethylene. It is in fact well known that the reactivity of the olefins decreases as the length of their carbon chain increases (C2>>C3>C4). The best-known systems for converting butenes by oligomerization are nickel-based systems of the Ziegler type. These systems convert butene-1 and butene-2 to a mixture of octenes (for reference, see “Reaction of unsaturated compounds”, p 240-253 in “Applied Homogeneous Catalysis with Organometallics” Ed. B. Cornils, W. Herrmann, Wiley-VCH, 2002).

The iron-based systems according to the invention therefore differ from the systems known from the literature. It is in fact surprising that the catalytic system reacts a little, if at all, with butene-2.

The purpose of the present invention is therefore to propose a method for separating butene-2 from a C4 feedstock comprising butene-1 and butene-2, characterized by the following successive steps:
a step of selective oligomerization of butene-1 by an iron-based catalytic composition
a step of separation of the oligomers formed from the butene-2.

The method according to the invention thus allows on the one hand the efficient separation of butene-2 from butene-1 and on the other hand the simultaneous production of predominantly linear octenes which can be used advantageously for example as raw material for hydroformylation for producing precursors for the production of plasticizers for polyvinyl chloride (PVC). The method of separation according to the invention in particular has the advantage of being a simple process that can be carried out with fewer steps than the methods of the state of the art such as that described in EP0079679.

Other separation steps can be added to the oligomerization step in the case of a feedstock containing, in addition to butene-1 and butene-2, other C4 components such as isobutene and/or 1,3-butadiene and/or acetylenic impurities.

In the case where the feedstock additionally contains isobutene, a step of separation of the isobutene is preferably carried out before the step of selective oligomerization.

In the case where the feedstock contains 1,3-butadiene and traces of acetylene hydrocarbons, a step of separation of the 1,3-butadiene and of the traces of acetylene hydrocarbons by selective hydrogenation is preferably carried out before the step of separation of the isobutene (optional) and/or before the step of selective oligomerization.

Thus, according to one variant, the method of separating butene-2 from a C4 feedstock containing butene-1, butene-2, isobutene, 1,3-butadiene and acetylenic impurities advantageously comprises the following successive steps:
an optional step of selective hydrogenation of 1,3-butadiene and acetylenic impurities,
an optional step of separation of the isobutene by etherification, dimerization or polymerization,
a step of selective oligomerization of butene-1 leading to separation of the butene-2 and to the co-production of predominantly linear oligomers.

This succession of steps offers numerous advantages. The most reactive compounds in the cut, namely 1,3-butadiene and traces of acetylene hydrocarbons, are converted in the very first step, and therefore will not cause parasitic reactions in the subsequent steps. Moreover, the selective hydroisomerization of 1,3-butadiene makes it possible to increase the concentration of butene-2, promoting the yield of this required product. The optional separation of the isobutene makes it possible to obtain isobutene that can be used in the production of polyisobutene, diisobutene or high-purity isobutene.

### DETAILED DESCRIPTION

The Feedstock

The feedstock used is a C4 hydrocarbon cut containing butene-1 and butene-2. It can additionally contain isobutene and/or 1,3-butadiene and/or acetylenic impurities. Typically, it contains butenes (butene-1 and butene-2), isobutene, paraffins (isobutane and n-butane), 1,3-butadiene and traces of acetylene hydrocarbons. These feedstocks can originate from a catalytic cracking unit, for example of the fluidized bed type, from a unit for steam cracking (of naphtha), and/or from a unit for dehydration of alcohols or from other sources.

Depending on the various components of the feedstock, one or more separation steps can advantageously precede the step of separation of butene-2 by selective oligomerization of butene-1. There may in particular be a step of separation of 1,3-butadiene and acetylenic impurities by selective hydrogenation and a step of separation of isobutene. Since these steps precede the oligomerization step, they will be described below, before the description of the step of selective oligomerization.

Selective Hydrogenation

The feedstock containing, in addition to butene-1 and butene-2, 1,3-butadiene and traces of acetylene hydrocarbons can be subjected to a step of selective hydrogenation.

The main aim of this optional first step is to convert 1,3-butadiene to butenes. The second aim of this step is to remove the traces of acetylene hydrocarbons still present in these cuts, which are poisons or contaminants for the subsequent steps. In fact, this step makes it possible to convert the most reactive compounds of the cut, namely 1,3-butadiene and the acetylene compounds in the very first step, and therefore they will not cause parasitic reactions in the subsequent steps. Moreover, this reaction makes it possible to increase the yield of n-butenes.

Another reaction involved in selective hydrogenation is the isomerization of butene-1 to butene-2 in thermodynamic equilibrium.

In this optional first step of selective hydrogenation, the following reactions are therefore carried out simultaneously:
selective hydroisomerization of 1,3-butadiene to a mixture of n-butenes in thermodynamic equilibrium,
isomerization of butene-1 to butene-2, also in thermodynamic equilibrium,
hydrogenation of the traces of acetylene hydrocarbons.

In the case of a feedstock originating from a steam cracker, preferably a step of selective hydrogenation of the dienes is carried out. In the case of a feedstock originating from a catalytic cracking unit, for example of the fluidized bed type, the step of selective hydrogenation of the dienes is optional. If the concentration of diolefins or acetylene in the cut used for said method is greater than 1000 ppm, selective hydrogenation is recommended in order to reduce this concentration to below 1000 ppm. Feedstocks containing more than 300 ppm of these impurities or even more than 10 ppm will preferably be treated in this step.

The reactions can be carried out by means of various specific catalysts comprising one or more metals, for example from group 10 of the periodic table (Ni, Pd, Pt), deposited on a support. Preferably a catalyst is used that comprises at least one palladium compound fixed on a refractory mineral support, for example on an alumina. The content of palladium on the support can be comprised between 0.01 and 5 wt. %, preferably between 0.05 and 1 wt. %. Various forms of pretreatment known to a person skilled in the art can optionally be applied to these catalysts to improve the selectivity in the hydrogenation of 1,3-butadiene to butenes at the expense of complete hydrogenation to butane, which must be avoided. The catalyst preferably contains 0.05 to 10 wt. % of sulphur. Advantageously, a catalyst constituted by palladium deposited on alumina, and containing sulphur, is used.

The sulphurization of the catalyst can be carried out in situ (in the reaction zone) or better still ex situ. In the latter case, the catalyst has advantageously been treated, before being loaded into the hydrogenation reactor, with at least one sulphur-containing compound diluted in a solvent. The catalyst obtained, containing 0.05 to 10 wt. % of sulphur, is loaded into the reactor and activated under a neutral or reducing atmosphere at a temperature comprised between 20 and 300° C., a pressure comprised between 0.1 and 5 MPa and an LHSV comprised between 50 and 600 h⁻¹, and the feedstock is brought into contact with said activated catalyst.

The utilization of a catalyst, preferably a palladium catalyst, is not critical, but it is generally preferable to use at least one reactor with descending flow through a fixed catalyst bed. When there is a high proportion of 1,3-butadiene in the cut, which is the case for example with a cut from steam cracking when extraction of the 1,3-butadiene for specific uses is not desired, it may be advantageous to carry out the conversion in two reactors in series, for better control of the hydrogenation selectivity. The second reactor can have ascending flow and can have a finishing role.

The quantity of hydrogen required for all of the reactions carried out in this step is adjusted depending on the composition of the cut so that there is, advantageously, only a slight excess of hydrogen relative to the theoretical stoichiometry.

The operating conditions are chosen so that the reagents and the products are in the liquid phase. It may, however, be advantageous to choose an operating mode such that the products are partially vaporized at the reactor outlet, which facilitates thermal control of the reaction. The temperature can vary from 20 to 200° C., preferably from 50 to 150° C. or more preferably from 60 to 150° C. The pressure can be adjusted between 0.1 and 5 MPa, preferably between 0.5 and 4 MPa and advantageously between 0.5 and 3 MPa, in such a way that the reagents are, at least partly, in the liquid phase. The space velocity can be comprised between 0.5 and 10 h⁻¹ and preferably between 1 and 6 h⁻¹, with an H₂/diolefins (molar) ratio from 0.5 to 5 and preferably from 1 to 3.

Preferably, selective hydrogenation of 1,3-butadiene and traces of acetylene hydrocarbons is carried out using a catalyst comprising at least one metal chosen from the group formed by nickel, palladium and platinum, deposited on a support, at a temperature of 20-200° C., a pressure of 1-5 MPa, a space velocity of 0.5-10 h⁻¹ and with an H₂/1,3-butadiene (molar) ratio from 0.5 to 5.

The hydroisomerization reactor or reactors can advantageously be followed by a stabilizing column which removes the excess traces of hydrogen and any methane.

Separation of Isobutene

The feedstock, containing isobutene in addition to butene-1 and butene-2, can be subjected to a step of separation of the isobutene. This optional step preferably takes place after the optional step of selective hydrogenation but before the oligomerization step.

In general, separation of the isobutene can be carried out by at least one of the following steps:
etherification of the isobutene to an alkyl tert-butyl ether in the presence of an etherification catalyst with an alcohol the hydrocarbon chain of which can have from 1 to 10 carbon atoms,
dimerization of the isobutene in the presence of an acid catalyst,
polymerization of the isobutene in the presence of a Lewis acid catalyst,

leading respectively most typically to MTBE (methyl tert-butyl ether), diisobutene or PIB (polyisobutene).

Preferably, separation of the isobutene is carried out by etherification.

Etherification

The feedstock containing butene-1, butene-2 and isobutene, and preferably after removal of 1,3-butadiene and traces of acetylene hydrocarbons, can be subjected to etherification in order to form a tertiary alkyl ether by contacting in a reaction zone containing an etherification catalyst. The etherification step is known to a person skilled in the art.

The aim of the second optional step is to convert the isobutene present in the C4 cut to an alkyl tert-butyl ether by etherification with an alcohol the hydrocarbon chain of which can comprise from 1 to 10 carbon atoms. Methanol or ethanol is preferably used as the alcohol. The ethers produced are methyl tert-butyl ether (MTBE) and ethyl tert-butyl ether (ETBE), respectively.

The conversion is carried out by means of an acid catalyst, for example a catalyst based on an ion-exchange resin in the H⁺ form, such as a sulphonic resin —SO₃H. This catalyst can be used for example conventionally in a fixed bed, or in a moving bed. It is preferable to work with an expanded bed of catalyst, maintained by a circulation with ascending flow of the reaction mixture through the reactor and an external heat exchanger. This manner of operating makes it possible to easily monitor the functioning of the catalyst as well as remove the heat produced in the reaction, avoiding the formation of hot spots.

A finishing reactor can advantageously be installed after the expanded-bed reactor for maximum exhaustion of the isobutene in the residual cut and to increase the yield of ether, but the major part of the reaction takes place in the expanded-bed reactor. Maximum exhaustion of the isobutene can also be achieved in the finishing process, by using a reactive distillation (distillation column containing catalyst), which makes it possible to increase the conversion by means of the separation of the products in the reactor.

The operating conditions are chosen in such a way that the reagents and the products are in the liquid phase. The temperature is generally set so that the reaction takes place at a sufficient rate, for example from 30 to 130° C., preferably from 40 to 100° C., and the pressure is consequently adjusted in such a way that the reagents are in the liquid phase.

Fractionation of the product originating from the etherification step makes it possible to obtain on the one hand an organic fraction enriched in tertiary alkyl ether and on the other hand an organic fraction depleted of tertiary alkyl ether containing butene-1 and butene-2 and optionally butanes, if present. Thus, the reaction section is followed by a distillation section where the ether is separated at the bottom of the column and a distillate comprising the residual C4 cut and the excess alcohol as well as traces of other volatile oxygen-containing compounds. The alcohol is separated by washing with water and the water-alcohol mixture is distilled to recycle the alcohol.

The ethers thus produced such as MTBE or ETBE can be used as additives for increasing the octane number in reformulated gasolines. It is also possible to recover the isobutene from the ethers produced, which are easily decomposed in the presence of an acid catalyst to release the purified isobutene.

Dimerization of Isobutene

The feedstock containing butene-1, butene-2 and isobutene, and preferably after removal of 1,3-butadiene and traces of acetylene hydrocarbons, can also be subjected to dimerization of isobutene to “di-isobutene”, a mixture of isomers containing mainly 2,2,4-trimethylpentene (isooctane). The step of dimerization of isobutene is known to a person skilled in the art.

This reaction is carried out in the presence of an acid catalyst, for example sulphuric acid, or acidic ion exchange resins, at temperatures between 20 and 130° C., preferably between 60 and 130° C. and even more preferably between 90 and 110° C.

This reaction can also be carried out using, as catalyst and as solvent, a composition comprising at least one Brønsted acid, designated HB, dissolved in an ionic liquid medium comprising at least one organic cation Q+ and an anion A⁻, as described in patent U.S. Pat. No. 7,256,152.

Polymerization of Isobutene

The feedstock containing butene-1, butene-2 and isobutene, and preferably after removal of 1,3-butadiene and traces of acetylene hydrocarbons, can also be subjected to polymerization of isobutene to polyisobutene (PIB), which can be included in the composition of elastomers, or also to liquid polybutenes which can be used as additives in lubricants. The step of polymerization of isobutene is known to a person skilled in the art.

The polymerization is carried out by mean of a Lewis acid catalyst, for example aluminum chloride, a chloroalkylaluminium, tin tetrachloride, boron trifluoride, optionally combined with traces of Brønsted acids such as hydrochloric acid, water, tert-butyl chloride, organic acids. The catalyst can be used in the solid state as powder or in the form of a suspension in a saturated hydrocarbon such as hexane or isobutane, or in a halogenated hydrocarbon such as methyl chloride.

The operating conditions are chosen in such a way that the reaction temperature can be controlled accurately. In general the temperature is set so that the reaction takes place at a sufficient rate, for example from −100 to +100° C., preferably from −50 to +50° C., and the pressure is adjusted for example in such a way that the hydrocarbon optionally used is partially vaporized by the heat released by the polymerization. In the case where the catalyst is used in the solid state, the heat of reaction can be removed by circulating the reaction mixture through an external exchanger in a loop.

The reaction section is followed by a section in which the catalyst is separated from the effluent, for example by washing with soda followed by washing with water, then by a separation (for example distillation) section where the polymer is separated.

It may be beneficial, to improve the quality of the products, to extract the isobutene from the effluent resulting from the previous step before polymerization. This extraction can be carried out by any well-known method, for example simply by distillation, or by hydration of the isobutene to tert-butyl alcohol in the presence of for example sulphuric acid, followed by separation and dehydration of the alcohol to recover the isobutene, or also by reaction with methanol in the presence of an acidic ion exchange resin to convert the isobutene to methyl tert-butyl ether (MTBE), which is easily separated by distillation, then decomposed in the presence of an acid catalyst to release the purified isobutene.

The residual C4 cut thus obtained contains butene-2, butene-1 as well as optionally butanes. It is subjected to the step of selective oligomerization.

Selective Oligomerization

The feedstock containing butene-1 and butene-2, and preferably after removal of 1,3-butadiene and traces of acetylene hydrocarbons and optionally after removal of isobutene, is subjected to a step of selective oligomerization of butene-1 by iron-based catalytic compositions without significant reaction of butene-2.

This step leads mainly to the formation of octenes, predominantly linear. In the case of a feedstock containing C4s, the selectivity for dimers (octenes) is generally greater than 50%, preferably greater than 70% relative to the converted olefins. The selectivity for linear dimers (n-octenes) relative to all of the dimers formed (octenes) is generally greater than 50%, preferably greater than 60%.

The iron-based catalytic composition used in the step of selective oligomerization is composed of at least one iron precursor, at least one ligand of the formula described below, complexed or not complexed with the iron precursor, and optionally an activating agent.

The iron precursor has the general formula FeXₙ, X being an anionic group, for example a halide such as a chloride, a fluoride, a bromide or an iodide; or a hydrocarbon group, for example a methyl, a benzyl or a phenyl; a carboxylate, for example an acetate or an acetylacetonate; an oxide; an amide, for example a diethyl amide; an alkoxide, for example a methoxide, an ethoxide or a phenoxide; or a hydroxyl. Alternatively, X can be a non-coordinating or weakly coordinating anion, for example a tetrafluoroborate, a fluorinated aryl borate or a triflate. The anionic group X can be monoanionic or dianionic. The iron precursors can be hydrated or not, coordinated or not with a ligand.

By way of example, the iron precursors can be iron(II) chloride, iron(III) chloride, iron(II) fluoride, iron(III) fluoride, iron(II) bromide, iron(III) bromide, iron(II) iodide, iron(III) iodide, iron(II) acetate, iron(III) acetate, iron(II) acetylacetonate, iron(III) acetylacetonate, iron(II) octoate, iron(III) octoate, iron(II) 2-ethylhexanoate, iron(III) 2-ethylhexanoate, iron(II) triflate, iron(III) triflate, iron(III) nitrate. The iron precursors can be hydrated or not, coordinated or not with a ligand such as tetrahydrofuran for example.

The ligand has the general formula:

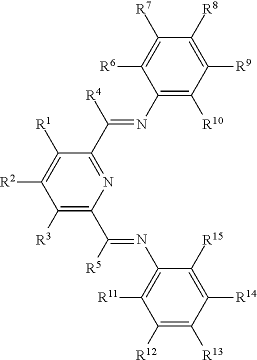

where R¹, R², R³, R⁴, R⁵, R⁶, R⁷, R⁸, R⁹, R¹⁰, R¹¹, R¹², R¹³, R¹⁴ and R¹⁵, which may be identical or different, are chosen from the hydrogen atom, linear or branched, cyclic or non-cyclic, saturated or unsaturated alkyl groups, aryl, aralkyl or alkaryl groups comprising 1 to 12 carbon atoms, groups containing hetero-elements, heterocyclic or not, aromatic or not, halides or not, supported or not, and preferably chosen from the alkoxy, nitro, halides and/or perfluoroalkyl group.

As non-limitative examples, R¹, R², R³, R⁴, R⁵, R⁶, R⁷, R⁸, R⁹, R¹⁰, R¹¹, R¹², R¹³, R¹⁴ and R¹⁵ can be chosen from hydrogen, methyl, ethyl, isopropyl, isobutyl, tert-butyl, cyclohexyl, phenyl, benzyl, methoxy, nitro, dimethylamine, diethylamine, diisopropylamine, trifluoromethyl, fluoride, chloride, bromide and iodide groups.

By way of example, the ligands can be of the following formula:

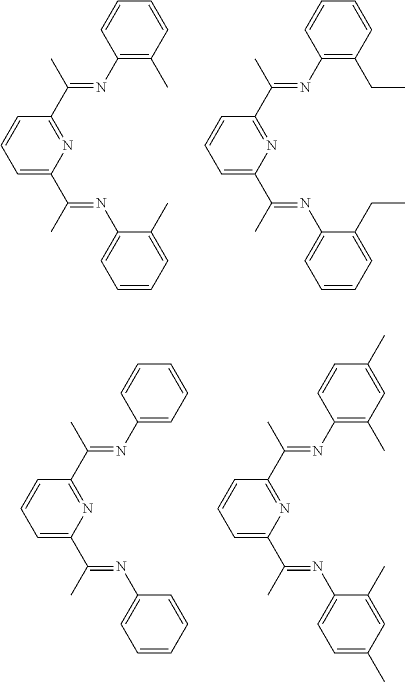 [SMILES: C/C(=N\c1ccccc1C)c1cccc(/C(C)=N/c2ccccc2C)n1 ; CCc1ccccc1/N=C(\C)c1cccc(/C(C)=N/c2ccccc2CC)n1 ; C/C(=N\c1ccccc1)c1cccc(/C(C)=N/c2ccccc2)n1 ; C/C(=N\c1ccc(C)cc1C)c1cccc(/C(C)=N/c2ccc(C)cc2C)n1]

By way of example, the complexes can be of the following formula:

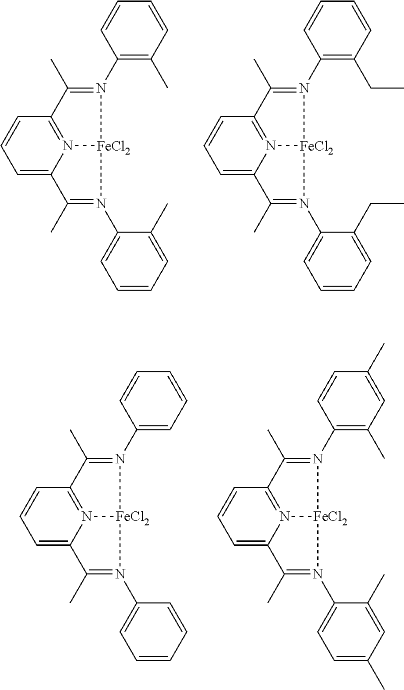

The optional activator for the catalyst used in the present invention is preferably a Lewis acid. Preferably, the Lewis acid is chosen from aluminum derivatives and boron or zinc derivatives or a mixture of these derivatives.

Examples of aluminum derivatives can include the alkylaluminiums, for example trimethylaluminium, triethylaluminium, tributylaluminium, tri-n-octylaluminium; alkylaluminium halides, for example diethylaluminium chloride, ethylaluminium dichloride, ethylaluminium sesquichloride, methylaluminium dichloride, isobutylaluminium dichloride; and aluminoxanes. The aluminoxanes are well known to a person skilled in the art as oligomeric compounds that can be prepared by controlled addition of water to an alkylaluminium, for example trimethylaluminium. Such compounds can be linear, cyclic or mixtures of these compounds. They are generally represented by the formula [RAlO]ₐ where R is a hydrocarbon group and “a” is a number from 2 to 50. Preferably, the aluminoxane is chosen from methylaluminoxane (MAO) and/or ethylaluminoxane (EAO) and/or from modified aluminoxanes such as modified methylaluminoxane (MMAO).

Examples of boron derivatives can include the trialkylboranes, for example trimethylborane, triethylborane, tripropylborane, tri-n-butylborane, tri-isobutylborane, tri-n-hexylborane, tri-n-octylborane, used alone or in a mixture, tris(aryl)boranes, such as for example tris(perfluorophenyl)borane, tris(3,5-bis(trifluoromethyl)phenyl)borane, tris(2,3,4,6-tetrafluorophenyl)borane, tris(perfluoronaphthyl)borane, tris(perfluorobiphenyl)borane and derivatives thereof. It is also possible to use, as activator, (aryl)borates associated with a triphenylcarbenium cation or with a trisubstituted ammonium cation such as triphenylcarbenium tetrakis(perfluorophenyl)borate, N,N-dimethylanilinium tetrakis(perfluorophenyl)borate, N,N-diethylanilinium tetrakis(3,5-bis(trifluoromethyl)phenyl)borate, triphenylcarbenium tetrakis(3,5-bis(trifluoromethyl)phenyl)borate.

Examples of zinc derivatives can include dialkylzincs, for example dimethylzinc, diethylzinc, dipropylzinc, dibutylzinc, dineopentylzinc, di(trimethylsilylmethyl)zinc, used alone or in a mixture.

Selective oligomerization of butene-1 can be carried out in the presence of a solvent. The organic solvent used in the catalytic composition will preferably be an aprotic solvent. Among the solvents that can be used in the method according to the present invention, aliphatic or cyclic hydrocarbons such as pentane, hexane, cyclohexane or heptane, aromatic hydrocarbons such as benzene, toluene or xylenes, chlorinated solvents such as dichloromethane or chlorobenzene, or acetonitrile, diethyl ether, tetrahydrofuran (THF) may be mentioned. The organic solvent will preferably be a saturated or unsaturated aliphatic solvent or an aromatic hydrocarbon.

The solvent can also be an ionic liquid, in a mixture or not with an organic solvent.

The reaction of selective oligomerization of butene-1 can be carried out in a closed system, in a semi-open system or continuously, with one or more reaction steps. Vigorous stirring must ensure good contact between the reagent or reagents and the catalytic composition.

The reaction temperature can be from −40 to +250° C., preferably from 0° C. to +150° C. The heat generated by the reaction can be removed by any means known to a person skilled in the art.

The reaction is preferably carried out in the liquid phase, and the pressure is adjusted to maintain the system in the liquid phase. Generally, the pressure can vary from atmospheric pressure to 10 MPa, and the pressure is preferably comprised between 5 and 8 MPa.

The effluent from selective oligomerization thus comprises the oligomers derived from butene-1, in particular predominantly linear octenes, butene-2 and optionally butanes, if present. This effluent is then subjected to a separation step, for example distillation, in order to separate the oligomers formed from the butene-2 (and from the butanes if present).

### BRIEF DESCRIPTION OF THE DRAWING

FIG. 1 is a diagrammatic representation of an advantageous embodiment according to the invention. The operating conditions have already been described and will not be repeated here.

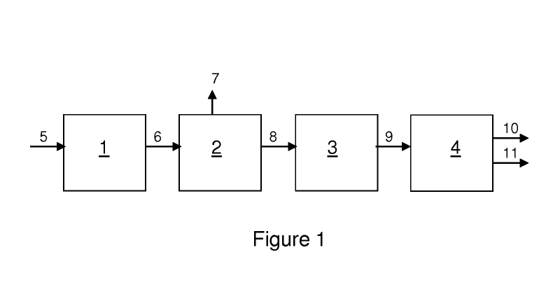

FIG. 1 describes a method of separating butene-2 from a C4 feedstock comprising, in addition to butene-2 and butene-1, isobutene, butadiene, n-butane, isobutane and traces of acetylene hydrocarbons; said method comprises the following successive steps:
an optional step of selective hydrogenation,
an optional step of separation of isobutene,
a step of selective oligomerization of butene-1,
then a step of separation of butene-2.

The C4 feedstock (5), which contains butene-1, butene-2, isobutene, butadiene, n-butane, isobutane and traces of acetylene hydrocarbons, is subjected to an optional step of selective hydrogenation (1) of 1,3-butadiene with isomerization of butene-1 to butene-2 in thermodynamic equilibrium and hydrogenation of the acetylene hydrocarbons. This step thus makes it possible to remove the 1,3-butadiene and traces of acetylene hydrocarbons. The effluent (6) containing butene-1, butene-2, isobutene, n-butane and isobutane is then sent to an optional step (2) of separation of isobutene, which can be carried out by etherification, by oligomerization or by selective polymerization of isobutene making it possible to obtain a stream (7) containing MTBE, diisobutene or PIB, and an effluent (8) containing butene-1, butene-2, n-butane and isobutane. This effluent (8) is then sent to a step of selective oligomerization of butene-1 (3) making it possible to obtain an effluent (9) containing predominantly linear oligomers (mainly octenes) in a mixture with butene-2, n-butane and isobutane. A step of separation (4), for example by distillation, then makes it possible to separate the oligomers (10) from a fraction (11) containing the unreacted butene-2 in a mixture with n-butane and isobutane.

### EXAMPLES

The following examples illustrate the invention without limiting its scope.

Preparation of the Catalyst Precursor:

The catalytic composition used is prepared as follows. The bis(imino)pyridine ligand (1.58 g, 1.05 eq.) and the precursor iron(II) dichloride tetrahydrate (0.88 g, 1 eq.) are added to a Schlenk flask under argon containing a magnetic bar. 150 mL of THF is then added and the solution is stirred at room temperature for 16 hours. The solid formed is isolated by filtration and then washed with ether and with pentane (2.17 g, 91%).

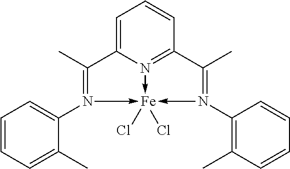

Catalytic Tests

The catalytic tests (Examples 1 and 2, Table 3) were carried out as follows: The 250-mL reactor is dried under vacuum at 80° C. for 3 hours and then placed under argon. The feedstock is injected into the reactor and then cooled to 0° C. The iron complex (2.10⁻⁵ mol) in 3 mL of toluene is introduced, as well as the activator (MAO, Al/Fe=105, 10 wt. % in toluene). Stirring is started and the temperature setting is fixed at 40° C. After the desired reaction time, neutralization is carried out with H₂O. The conversions are monitored by GC analysis of the gas and liquid phases.

In Example 1 (not according to the invention) the feedstock is butene-1. In Example 2 (according to the invention), the feedstock is a mixture of butene-1 and butene-2 with a butene-1/butene-2 ratio of 0.85.

TABLE 3
Catalytic tests

| | | | | | Linear octenes |
|---|---|---|---|---|---|
| | | | Conversion | Dimers | (wt. % of n- |
| | | Duration | of | (wt. %/total | octenes/total |
| No. | Feedstock | (minutes) | butene-2 | products) | dimers) |
| 1 | butene-1 | 165 | 0 | 70 | 67 |
| 2 | butene-1 + | 360 | <5 | 73 | 65 |
| | butene-2 | | | | |

Example 2 shows that carrying out the method according to the invention makes it possible to separate butene-2 from a C4 cut containing butene-1 and butene-2 by selective oligomerization of butene-1 to oligomers with a selectivity for dimers of 73 wt. % and a linearity of the octenes formed of 65 wt. %/total. The conversion of the butene-2 remains extremely low. Comparison of Examples 1 and 2 shows that the presence of butene-2 has little effect on the selectivity and linearity.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited herein and of corresponding French application No. 11/02849, filed Sep. 20, 2011, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Method of separating butene-2 from a C4 feedstock comprising butene-1 and butene-2, characterized by the following successive steps:
a step of selective oligomerization of butene-1 by an iron-based catalytic composition
a step of separation of the oligomers formed from the butene-2.

2. Method according to claim 1 in which the iron-based catalytic composition comprises an iron precursor and at least one ligand of the formula described below, complexed or not complexed with the iron precursor, and said iron precursor can be hydrated or not and has the general formula FeXₙ, X being an anionic group chosen from a halide, a hydrocarbon group, a carboxylate, an oxide, an amide, an alkoxide, a hydroxide or a non-coordinating or weakly coordinating anion, said ligand having the general formula: 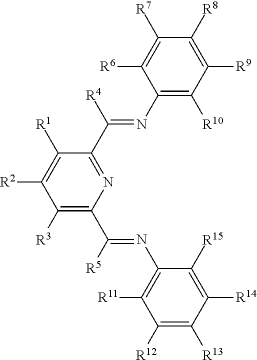
where R¹, R², R³, R⁴, R⁵, R⁶, R⁷, R⁸, R⁹, R¹⁰, R¹¹, R¹², R¹³, R¹⁴ and R¹⁵, which may be identical or different, are chosen from the hydrogen atom, linear or branched, cyclic or non-cyclic, saturated or unsaturated alkyl groups, aryl, aralkyl or alkaryl groups comprising 1 to 12 carbon atoms, groups containing hetero-elements, heterocyclic or not, aromatic or not, halides or not, supported or not.

3. Method according to claim 1 in which the catalytic composition comprises an activator chosen from aluminum derivatives and boron or zinc derivatives or a mixture of said derivatives.

4. Method according to claim 1 in which the activator is chosen from the alkylaluminiums, alkylaluminium halides, and aluminoxanes, trialkylboranes, tris(aryl)boranes, (aryl)borates associated with a triphenylcarbenium cation or with a trisubstituted ammonium cation, dialkylzincs.

5. Method according to claim 3 in which the activator is an aluminoxane chosen from methylaluminoxane (MAO) and/or ethylaluminoxane (EAO) and/or from modified aluminoxanes such as modified methylaluminoxane (MMAO).

6. Method according to claim 1 in which the step of selective oligomerization is carried out in the presence of an organic solvent chosen from aliphatic or cyclic hydrocarbons, aromatic hydrocarbons, chlorinated solvents, acetonitrile, diethyl ether and/or tetrahydrofuran, and/or an ionic liquid solvent.

7. Method according to claim 1 in which the step of selective oligomerization is carried out at a temperature between −40 and +250° C. and at a pressure varying from atmospheric pressure to 10 MPa.

8. Method according to claim 1 in which the feedstock also contains isobutene and a step of separation of the isobutene is carried out before the oligomerization step.

9. Method according to claim 1 in which the separation of isobutene is carried out by at least one of the following steps:
etherification of isobutene to an alkyl tert-butyl ether in the presence of an etherification catalyst with an alcohol the hydrocarbon chain of which can comprise from 1 to 10 carbon atoms,
dimerization of the isobutene in the presence of an acid catalyst,
polymerization of the isobutene in the presence of a Lewis acid catalyst.

10. Method according to claim 1 in which the feedstock also contains 1,3-butadiene and optionally traces of acetylene hydrocarbons and a step of separation of butadiene and any traces of acetylene hydrocarbons by selective hydrogenation is carried out before the step of separation of isobutene and/or the step of selective oligomerization.

11. Method according to claim 1 in which the selective hydrogenation of 1,3-butadiene and traces of acetylene hydrocarbons is carried out with a catalyst comprising at least one metal chosen from the group formed by nickel, palladium and platinum, deposited on a support, at a temperature of 20-200° C., a pressure of 1-5 MPa, a space velocity of 0.5-10 h⁻¹ and with an H₂/butadiene (molar) ratio of 0.5 to 5.

12. Method according to claim 1 in which the feedstock originates from a catalytic cracking unit, from a steam cracking unit and/or from an alcohol dehydration unit.
